(19) United States
(12) Patent Application Publication
(10) Pub. No.: US 2008/0051443 A1
(43) Pub. Date: Feb. 28, 2008

(54) Compounds for the reduction of excessive food intake

(76) Inventors: Michael Paul Pieper, Biberach (DE); Joachim Mierau, Mainz (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/857,016

(22) Filed: Sep. 18, 2007

Related U.S. Application Data
(63) Continuation of application No. 10/935,508, filed on Sep. 7, 2004, now abandoned.
Application No. 10/935,508 is a continuation of application No. 10/259,118, filed on Sep. 27, 2002, now abandoned.

(30) Foreign Application Priority Data
Sep. 28, 2001 (DE) 101 48 233.7

(51) Int. Cl. A61K 31/426; A61P 3/04

(52) U.S. Cl. 514/367; 514/789

(57) ABSTRACT

The invention relates to the use of dopamine receptor agonists for preparing a pharmaceutical composition for the reduction of excessive food intake.

### RELATED APPLICATIONS

This application is a continuation of U.S. application Ser. No. 10/935,508, filed Sep. 7, 2004, which is a continuation of U.S. application Ser. No. 10/259,118, filed Sep. 27, 2002 which claims priority benefit, as does the present application, to German Application No. 101 48 233.7, filed Sep. 28, 2001.

### BACKGROUND

The invention relates to the use of dopamine receptor agonists for preparing a pharmaceutical composition for reducing excessive food intake. The invention relates to pharmaceutical compositions for treating overweight or obesity by reducing food intake comprising administering a dopamine receptor agonist and methods for treating overweight or obesity comprising administering a pharmaceutical composition of the invention such that food intake is reduced.

### SUMMARY

Excessive food intake generally leads to overweight or obesity, i.e. an increase in normal weight which exceeds normal limits. Nowadays, being overweight is not only a serious health risk but also an economic problem. Overweight is a risk factor for a number of diseases such as high blood pressure, diabetes mellitus, hyperlipidemia, osteoarthritis, gout and the associated vascular diseases, particularly arteriosclerosis. Moreover, being overweight can cause emotional problems including depression.

The only effective therapeutic action is to reduce calorie intake. However, this is very difficult to achieve in many patients in spite of a knowledge of the consequences mentioned above.

The objective of the invention is to make it easier for the patient to reduce their calorie consumption and thus reduce the health risks associated with overweight or obesity.

### FIGURES

FIG. 1: 4-day long-term administration of pramipexole (PPX) compared with a single administration on 4 successive days. Test group A is Alzet pump NaCl 0.9% s.c.; B is Alzet pump PPX 2.5 mg/24 h s.c.; C is NaCl 0.9% s.c., single administration on 4 successive days; D is PPX 2.5 mg/kg s.c., single administration on 4 successive days. Significance versus control group: *p<0.05; **p<0.01; ***p<0.001.

FIG. 2: Reduction in body weight during 14 days of continuous subcutaneous (s.c.) infusion of pramipexole and 7 days subsequent observation. Significance versus control group: *p<0.05; **p<0.01; ***p<0.001.

### DETAILED DESCRIPTION OF THE INVENTION

It can now be shown that, surprisingly, dopamine receptor agonists selected from among the D₁, D₂, D₃ and D₄ receptor agonists may be used to good effect in therapeutically effective doses to reduce excess food intake.

Accordingly, the present invention relates to the use of dopamine receptor agonists, selected from among D₁, D₂, D₃ and D₄ receptor agonists, for preparing a pharmaceutical composition for the reduction of excessive food intake. Preferably, dopamine receptor agonists are selected from among adrogolide, A-86929, rotigotine, NeurVex, Nolomirole, pramipexole, talipexole, CHF 1512, (−)-stepholidine, DAR-201, Diacrin/Genzyme, bromocriptine, Bupropion, LEK-8829, BAM-1110, AIT-203, NS-2330, Terguride, Aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, ropinirole, SKF-82958, SKF-77434, DU 127090, SLV-308, SLV 318, NeuroCRIB, SP-1037C, spheramine, Gallotrank, Preclamol, DAB-452, YM-435, BP-897, ProSavin, Etilevodopa, P63, A 68930, A 77636, Alaptide, Alentemol, CI 1007; PD 143188, BLSI, JA 116a; JA 116, Melevodopa; Levodopa methyl; CHF 1301; NSC 295453; Levomet, MR 708, PD 128483, RD 211, SKF 38393, SKF 81297, U 86170F, U 91356A, WAY 124486 and Z 15040.

The invention encompasses pharmaceutical compositions for treating overweight or obesity by reducing food intake comprising administering a dopamine receptor agonist, preferably a D₃ receptor agonist, more preferably pramipexole or talipexole.

The invention also encompasses methods for treating overweight or obesity comprising administering a dopamine receptor agonist, preferably a D₃ receptor agonist, more preferably pramipexole or talipexole such that food intake is reduced.

In an embodiment, the methods for treating overweight or obesity encompass treating obesity in type 2 diabetes.

The invention also encompasses methods for reducing food intake comprising administering a dopamine receptor agonist, preferably a D₃ receptor agonist, more preferably pramipexole or talipexole such that food intake is reduced.

It is particularly preferable to use the above-mentioned dopamine receptor agonists to prepare a pharmaceutical composition for the reduction of excessive food intake in cases of overweight or obesity.

It is also preferred to use the above-mentioned dopamine receptor agonists to prepare a pharmaceutical composition for treating obesity in type 2 diabetes.

It is particularly preferred to use the above-mentioned dopamine receptor agonists to prepare a pharmaceutical composition for continuous administration for the reduction of excessive food intake.

It is most preferred to use the above-mentioned dopamine receptor agonists to prepare a pharmaceutical composition for transdermal administration for the reduction of excessive food intake.

Pramipexole and talipexole are particularly preferred as they have high selectivity for the dopamine D₃ receptor. It can be demonstrated that this reduces the side effects of any pharmacological control of food intake. The D₃ receptor is located predominantly in those regions of the brain which are associated with emotion. Activation of the D₃ receptor by a dopamine agonist, preferably pramipexole or talipexole, particularly preferably by pramipexole, may contribute to a reduction in excessive food intake or pathologically disturbed food intake by lightening the patient's mood.

The dopamine D₃ receptor agonists pramipexole and talipexole which are preferably used within the scope of the present invention may optionally be used in the form of their enantiomers or racemates, optionally in the form of the pharmacologically acceptable acid addition salts, and optionally in the form of the hydrates and solvates.

Of exceptional importance within the scope of the application according to the invention is the dopamine D₃ receptor agonist pramipexole, optionally in the form of its enantiomer or racemate, optionally in the form of the pharmacologically acceptable acid addition salts, and optionally in the form of the hydrates and solvates.

Any reference to one of the above-mentioned dopamine D₃ receptor agonists includes a reference to any enantiomer or racemate of the compound which may exist. For example, a reference to pramipexole includes a reference to the (+)-enantiomer and to the racemate. However, within the scope of the present invention, the (−)-enantiomer is of particular significance.

The dopamine D₃ receptor agonists which may be used according to the invention may optionally be used in the form of the pharmaceutically acceptable acid addition salts thereof and optionally in the form of the hydrates and solvates.

By the “pharmaceutically acceptable acid addition salts” of the dopamine D₃ receptor agonists are meant, according to the invention, those salts which are selected from among the salts of hydrochloric acid, hydrobromic acid, sulphuric acid, phosphoric acid, methanesulfonic acid, acetic acid, fumaric acid, succinic acid, lactic acid, citric acid, tartaric acid and maleic acid, the salts of hydrochloric acid, hydrobromic acid, sulphuric acid, phosphoric acid and acetic acid being particularly preferred. The salts of hydrochloric acid are of particular significance.

In the case of pramipexole, the use of which is particularly preferred according to the invention, the hydrochlorides are preferably used, while pramipexole dihydrochloride is of particular importance. For transdermal administration it is preferable to use the base of pramipexole. Of the hydrates of pramipexole, pramipexole dihydrochloride monohydrate is particularly preferred.

The dopamine receptor agonists, preferably dopamine D₃ receptor agonists, preferably pramipexole or talipexole, most preferably pramipexole, which may be used according to the invention may optionally be used in conjunction with other active substances. Preferred combination partners are compounds selected from the categories of the dopamine D₁, D₂, D₃, or D₄ receptor agonists, selected from among Adrogolide, A-86929, Rotigotine, NeurVex, Nolomirole, pramipexole, talipexol, CHF 1512, (−)-Stepholidine, DAR-201, Diacrin/Genzyme, Bromocriptine, Bupropion, LEK-8829, BAM-1110, AIT-203, NS-2330, Terguride, Aripiprazole, OPC-4392, GMC-1111, PD-148903, Apomorphine HCl, PD-89211, PD-158771, Cabergoline, Sumanirole, PNU-14277E, POL-255, Dihydrexidine, GBR-12783, Quinagolide HCl, (R)-Bupropion, S-32504, S-33592, SKF-80723, SKF-83959, Fenoldopam, Ropinirole, SKF-82958, SKF-77434, DU 127090, SLV-308, SLV 318, NeuroCRIB, SP-1037C, Spheramine, Gallotrank, Preclamol, DAB-452, YM-435, BP-897, ProSavin, Etilevodopa, P63, A 68930, A 77636, Alaptide, Alentemol, CI 1007; PD 143188, BLSI, JA 116a; JA 116, Melevodopa; Levodopa methyl; CHF 1301; NSC 295453; Levomet, MR 708, PD 128483, RD 211, SKF 38393, SKF 81297, U 86170F, U 91356A, WAY 124486 and Z 15040, the antidepressants, the anorectics, preferably silbutramin, the lipase inhibitors, preferably Orlistat, and the sympathomimetics, preferably ephedrine. The dosages of the individual components can be reduced, due to the synergistic effects obtained when combinations containing one of the additional active substances in addition to the dopamine receptor agonists according to the invention are used as envisaged.

The novel activity of the dopamine agonists according to the invention is illustrated by means of the following Example using pramipexole. It serves merely to illustrate the invention and is not to be regarded as limiting.

### EXAMPLE

Effect of Pramipexole on Food Intake in Mice:

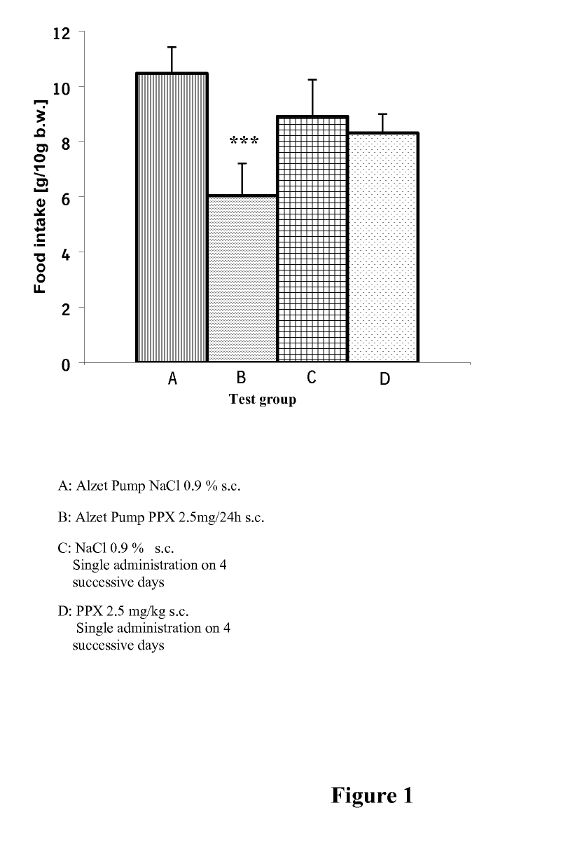

When administered over the longer term pramipexole inhibited food intake in mice. Long-term administration using osmotic pumps led to a permanent, statistically highly significant inhibition of food intake (FIG. 1). In contrast, a single application given on successive days in a dosage comparable to that of the long-term administration did not lead to any significant reduction in food intake (FIG. 1).

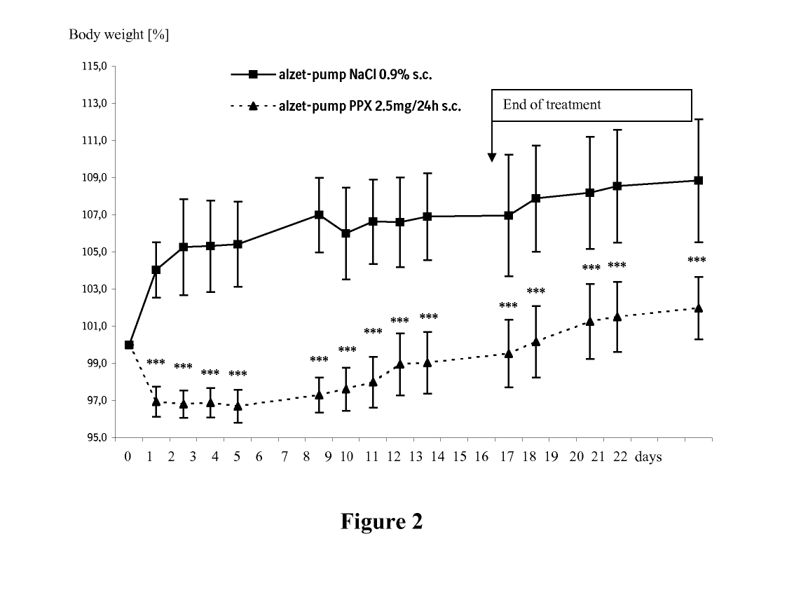

Moreover, with long-term administration, a permanent reduction in weight was observed which was statistically highly significantly detectable even after the pramipexole treatment had ended (FIG. 2).

Test Method Using Single Administration:

Ten mice (strain: C57BL/6) were deprived of food for 24 h while having free access to drinking water.

Twenty minutes before the end of the fasting period the mice were given pramipexole (2.5 mg/kg of body weight s.c.). The control group, 10 mice in each case, were given physiological saline solution, the solvent used for pramipexole. Then the animals were offered food and their food consumption was measured over 4 days at intervals of 30 min.

Test Method Using Long-Term Administration:

Ten mice (strain: C57BL/6) were deprived of food for 24 h while having free access to drinking water.

Twenty minutes before the end of the fasting period an alzet® Mini-osmotic pump (model 2002) releasing a dose of 2.5 mg of pramipexole/24 hours s.c. was implanted subcutaneously in the animals. The pumping rate was 0.54 μl/h. A group of 10 control animals were given the solvent, physiological saline solution, at the same pumping rate, in an analogous test. The continuous release of the substance or solvent was measured for 4 days. The food intake was measured at 2 hour intervals over the first ten hours, and later daily.

The change in weight with long-term administration was measured over a period of 22 days, the administration of pramipexole being terminated after 14 days. The change in weight was measured each day.

The dosage of the dopamine receptor agonists according to the invention is naturally highly dependent on the severity of the symptoms to be treated on the one hand and the choice of active substance on the other hand. For example, without restricting the subject matter of the present invention thereto, some possible dosages especially for the compound pramipexole which is particularly preferred according to the invention include but are not limited to the following dosages of: about 0.05 to 3 mg, preferably about 0.1 to 1.5 mg per day. These dosages are based on pramipexole in the form of its free base. Based on the salt form which is preferably used, namely pramipexole dihydrochloride monohydrate, the above-mentioned dosages correspond to about 0.07 to 4.26 mg, preferably 0.14 to 2.13 mg of pramipexole dihydrochloride monohydrate per day.

One possible dosing method, which is described solely as an illustrative example, is described hereinafter (based on pramipexole in the form of its free base): individual dosage titration at weekly intervals depending on the activity and tolerance levels.

1st week: 1 tablet containing 0.088 mg of pramipexole 3 times a day;

2nd week: 1 tablet containing 0.18 mg of pramipexole 3 times a day;

3rd week and thereafter: ½ tablet containing 0.7 mg of pramipexole 3 times a day.

The dopamine D₃ receptor agonists may be administered for the purposes according to the invention by oral, transdermal, intrathecal, inhalative, nasal or parenteral route, preferably by transdermal or parenteral route, most preferably by transdermal route. Suitable preparations include, for example, tablets, particularly slow release tablets, capsules, suppositories, solutions, syrups, emulsions, dispersible powders, implants or plasters, most preferably micronal plasters. In connection with possible embodiments of a transdermal preparation which may be used according to the invention reference is hereby made, particularly with regard to pramipexole, to the embodiments according to U.S. Pat. No. 5,112,842, the contents of which are expressly noted at this point. Tablets may be obtained, for example, by mixing the active substance or substances with known excipients, e.g. inert diluents such as calcium carbonate, calcium phosphate or lactose, disintegrants such as maize starch or alginic acid, binders such as starch or gelatine, lubricants such as magnesium stearate or talc, and/or agents for obtaining delayed release such as carboxymethylcellulose, cellulose acetate phthalate, or polyvinylacetate. The tablets may also consist of several layers.

Some examples of pharmaceutical preparations which may be used according to the invention are given below. These are intended solely as illustrations by way of example without restricting the subject matter of the invention thereto.

Tablet 1:

| Ingredients: | mg |
|---|---|
| Pramipexole dihydrochloride monohydrate | 1.00 |
| Mannitol | 121.50 |
| Maize starch | 79.85 |
| Highly dispersed silicon dioxide, anhydrous | 2.30 |
| Polyvidon K25 | 2.35 |
| Magnesium stearate | 3.00 |
| Total | 210.00 |

Tablet 2:

| Ingredients: | mg |
|---|---|
| Pramipexole | 0.5 |
| Mannitol | 122.0 |
| Maize starch, dried | 61.8 |
| Maize starch | 18.0 |
| Highly dispersed silicon dioxide, anhydrous | 2.4 |
| Polyvidon K25 | 2.3 |
| Magnesium stearate | 3.0 |
| Total | 210.0 |

Tablet 3:

| Ingredients: | mg |
|---|---|
| Pramipexole | 0.25 |
| Mannitol | 61.00 |
| Maize starch | 39.90 |
| Highly dispersed silicon dioxide, anhydrous | 1.20 |
| Polyvidon K25 | 1.15 |
| Magnesium stearate | 1.5 |
| Total | 105.00 |

Tablet 4:

| Ingredients: | mg |
|---|---|
| Pramipexole | 0.125 |
| Mannitol | 49.455 |
| Maize starch, dried | 25.010 |
| Maize starch | 7.300 |
| Highly dispersed silicon dioxide, anhydrous | 0.940 |
| Polyvidon K25 | 0.940 |
| Magnesium stearate | 1.230 |
| Total | 85.000 |

Solution for injection:

| | | |
|---|---|---|
| pramipexol dihydrochloride monohydrate | 0.3 | mg |
| sodium chloride | 0.8 | mg |
| benzalkonium chloride | 0.01 | mg |
| water for injections | ad 100 | ml |

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various patent applications and publications are cited herein, the disclosures of which are incorporated by reference in their entireties.

What is claimed is:

1. A method for treating overweight or obesity comprising administering a pharmaceutical composition comprising a dopamine receptor agonist selected from the group consisting of: D₁, D₂, D₃, and D₄ receptor agonists, such that food intake is reduced.

2. A method for reducing food intake comprising administering a pharmaceutical composition comprising a dopamine receptor agonist selected from the group consisting of: D₁, D₂, D₃, and D₄ receptor agonists, such that food intake is reduced.

3. The method of claim 1 wherein the obesity is obesity in type 2 diabetes.

4. The method of claims 1 or 2 wherein the pharmaceutical composition is administered continuously.

5. The method of claims 1 or 2 wherein the pharmaceutical composition is administered transdermally.

6. The method of claim 1 wherein the dopamine receptor agonist is pramipexole or talipexole.

7. The method of claim 6 wherein the dopamine receptor agonist is pramipexole.

8. The method of claim 6 wherein the pramipexole or talipexole is administered in the form of an enantiomers.

9. The method of claim 6 wherein the pramipexole or talipexole is administered in the form of a pharmacologically acceptable acid addition salts.

10. The method of claim 6 wherein the pramipexole or talipexole is administered in the form of a hydrate.

11. The method of claim 6 wherein the pramipexole or talipexole is administered in the form of a solvate.
